(19) United States
(12) Patent Application Publication
(10) Pub. No.: US 2013/0102591 A1
(43) Pub. Date: Apr. 25, 2013

(54) COMPOSITIONS AND METHODS FOR PREVENTION AND TREATMENT OF PULMONARY HYPERTENSION

(71) Applicants: Andrew Lurie Salzman, West Tisbury, MA (US); Shlomo Magdassi, Jerusalem (IL); Katrin Margulis-Goshen, Jerusalem (IL)

(72) Inventors: Andrew Lurie Salzman, West Tisbury, MA (US); Shlomo Magdassi, Jerusalem (IL); Katrin Margulis-Goshen, Jerusalem (IL)

(21) Appl. No.: 13/575,388

(22) PCT Filed: Jan. 26, 2011
(86) PCT No.: PCT/IL2011/000087

§ 371 Date: Dec. 28, 2012

(30) Foreign Application Priority Data
Jan. 26, 2010 (US) 61298224
May 13, 2010 (US) 61334211

(51) Int. Cl. A61K 31/40; A61K 31/55; A61K 31/4545; A61K 31/445

(52) U.S. Cl. CPC A61K 31/40; A61K 31/445; A61K 31/55; A61K 31/4545; Y10S 977/773; Y10S 977/915; B82Y 5/00
USPC 514/217.12; 514/424; 514/315; 514/316; 514/425; 977/773; 977/915

(57) ABSTRACT

The invention provides compositions and methods for prevention, treatment, or management of pulmonary hypertension using piperidine, pyrrolidine, or azepane derivatives comprising one to four nitric oxide (NO) donor groups and a reactive oxygen species (ROS) degradation catalyst. The invention further provides a water dispersible powder comprising nanoparticles comprising said derivatives, as well as pharmaceutical compositions thereof and methods of use.

### TECHNICAL FIELD

The present invention relates to use of compounds comprising a nitric oxide (NO) donor and a reactive oxygen species (ROS) degradation catalyst in pharmaceutical compositions and methods for prevention, treatment, or management of pulmonary hypertension.

### BACKGROUND ART

Pulmonary hypertension (PH) is a severe disease characterized by increased pulmonary vascular resistance and pulmonary arterial pressure, and ultimately pulmonary vascular remodeling effects that interfere with ventilation-perfusion relationships and compromise ventricular function. The disease is defined by a mean pulmonary arterial pressure (MPAP)>25 mmHg at rest or >30 mmHg with exercise.

PH is currently classified into five groups, wherein pulmonary arterial hypertension (PAH) is classified as Group 1; PH associated with left heart diseases is classified as Group 2; PH associated with lung diseases and/or hypoxemia is classified as Group 3; PH due to chronic thrombotic and/or embolic diseases is classified as Group 4; and PH of other origin is classified as Group 5 (Galiè et al., 2004).

PAH includes both idiopathic and familial PAH as well as PAH associated with autoimmune connective tissue diseases such as scleroderma, CREST (calcinosis cutis, Raynaud phenomenon; esophageal motility disorder; sclerodactyl), and teleangiectasia), sarcoidoisis, systemic lupus erythematosus, and rheumatoid arthritis; hemoglobinopathies such as sickle cell disease, paroxysmal nocturnal hemoglobinuria, alpha and beta thalassemia, and glucose-6-phosphate dehydrogenase deficiency; bacterial (including mycoplasma), viral, fungal, or rickettsial pneumonia; acute lung injury secondary to aspiration or trauma; congenital systemic to pulmonary shunts, e.g., aorto-pulmonary window, persistent ductus arteriosus, truncus arteriosus, ventricular septal defect, or atrial septal defect; portal hypertension; HIV; and drug, e.g., anorexigens, or toxin intake. Muscularization of small (less than 500 μm diameter) pulmonary arterioles is widely accepted as a common pathological denominator of PAH; however, it may also occur in other forms of PH such as those associated with chronic obstructive pulmonary disease (COPD) or thrombotic and/or thromboembolic disease. Prominent anatomical features in PH include thickening of the vascular intima based upon migration and proliferation of (myo)fibroblasts or smooth muscle cells and excessive generation of extracellular matrix, endothelial injury, and/or proliferation and perivascular inflammatory cell infiltrates.

Despite its pleiotropic etiologies, the disease course of PH is inexorable, and if not treated, progresses to end-stage right ventricular failure (cor pulmonale). The prognosis for patients with primary PH is poor, with a median survival time of two to three years from diagnosis if untreated. Generally, progression of the disorder leads inexorably to syncope and right heart failure, and death is often sudden.

PH has multiple triggers; however, it is thought that all initiate biological cascades that converge on a final common effector mechanism of vascular and tissue injury produced by an excess of the oxygen-centered free radical superoxide and a deficiency of the nitrogen-centered free radical NO in the pulmonary vasculature. NO deficiency results both from its consumption by superoxide and its diminished synthesis by the endothelial NO synthase (ecNOS), secondary to depletion of its precursor (L-arginine) and synthetic co-factor tetrahydrabiopterin (BH4). Superoxide is correspondingly elevated due to its excessive production by uncoupled mitochondria, NADPH oxidase, xanthine oxidase, and uncoupled ecNOS, or, in the special case of the very low birthweight (VLBW) premature infant, as a result of the developmental absence of anti-oxidant defenses.

The Role of NO in PH:

In the acute PH setting, NO maintains the vasculature in a dilated state, free of platelet adhesion and activation, via the activation of the enzyme guanylate cyclase. In the chronic PH setting, NO blocks vascular hypertrophy and remodeling by triggering a biological cascade that regulates smooth muscle cell division. In particular, NO inactivates ornithine decarboxylase (ODC) in the vessel wall via its nitrosylation of a critical ODC cysteine residue, which in turn decreases ODC-mediated production of putrescine from ornithine. Depletion of putrescine triggers MAPK1/2-mediated activation of the cyclin dependent kinase inhibitor p21(waf1/cip1), and elevation of p21 activity arrests the G(1)→S phase cell cycle transition and thereby inhibits vascular smooth muscle cell proliferation.

The Role of Reactive Oxygen Species (ROS) in pH:

Under physiological conditions, the endothelium produces, in addition to NO, superoxide, and other ROS. Superoxide is the prime scavenger of NO and thus lowers NO concentration. In addition, superoxide induces vasoconstriction by opening L-type calcium channels and chemically combining with NO to yield peroxynitrite. Peroxynitrite attacks three key enzymes that mediate vasodilation in the lung: (i) guanylate cyclase, the biological target of NO that induces vasorelaxation via its generation of cGMP; (ii) ecNOS, which in its uncoupled state produces superoxide instead of NO; and (iii) prostacylin synthase (PGI2), an enzyme that produces prostacyclin, a vasodilating prostaglandin that increases cAMP. Peroxynitrite further induces DNA single strand breakage, resulting in activation of the DNA repair enzyme poly(ADP-ribose) polymerase, which then consumes NADPH and ATP, both required for endothelium-dependent smooth muscle relaxation. Peroxynitrite excess in the lung has been described in PH associated with hemolytic disease, autoimmunity, pneumonia, Adult Respiratory Distress Syndrome, and prematurity.

The imbalance of NO and superoxide directly impairs the ability of the pulmonary arteriole to dilate and conduct blood flow at a low pressure, and ultimately and irreversibly damages the vascular smooth muscle. More particular, superoxide excess and NO deficiency, when taken together, profoundly disrupt vascular smooth muscle physiology, resulting in pulmonary arteriolar vasoconstriction and hypertension, pulmonary vascular hypertrophy, right heart failure, and death. There is thus a need for restoring the NO-superoxide balance by simultaneously providing exogenous NO and removing endogenous superoxide.

U.S. Pat. No. 5,958,427 discloses certain NO donor compounds and pharmaceutical compositions comprising thereof for delivering NO to the apical surface of a mucosa, wherein NO is released for passage across the epithelial monolayer of the mucous membrane. The compounds disclosed include tertiary and quaternary amino aliphatic NO donor compounds as well as polyalkyleneamine nonoates, and are particularly useful for treatment of PH and male impotence. A potential concern with this therapy is the chemical reaction of exogenously administered NO with ambient superoxide, resulting in the formation of peroxynitrite, a powerful toxin.

U.S. Pat. No. 7,378,438 discloses β-agonist compounds comprising an ROS scavenger group and an NO donor, which are useful for treatment of respiratory diseases involving airway obstruction such as asthma and chronic bronchitis. Nevertheless, this patent neither teaches nor suggests the use of an NO donor together with an ROS degradation catalyst in general, and for treatment of PH in particular.

U.S. Pat. Nos. 6,448,267, 6,455,542 and 6,759,430, herewith incorporated by reference in their entirety as if fully described herein, disclose, inter alia, piperidine, pyrrolidine and azepane derivatives comprising an NO donor and a superoxide scavenger, capable of acting as sources of NO and as ROS degradation catalysts, their preparation, and their use in treatment of various conditions associated with oxidative stress or endothelial dysfunction.

### SUMMARY OF INVENTION

It has been found, in accordance with the present invention, that administration of certain 1-pyrrolidinyloxy derivatives, more particular, 3-nitratomethyl-2,2,5,5-tetramethylpyrrolidinyloxy, in a rat pulmonary hypertension model, starting 38 days after monocrotaline (MCT) administration and over a course of therapy of 10 days, significantly reduced both the elevation of pulmonary arterial hypertension (PAH) and the histological lung injury, i.e., alveolar damage, inflammatory cell infiltrate, and vascular smooth muscle hypertrophy that developes in response to MCT administration. These findings are of high significance since 3-nitratomethyl-2,2,5,5-tetramethylpyrrolidinyloxy therapy started after MCT injection, a timepoint when PAH and lung injury have already been established.

In one aspect, the present invention thus relates to a method for prevention, treatment or management of pulmonary hypertension (PH) in an individual in need thereof, comprising administering to said individual a therapeutically effective amount of a compound of the general formula I:

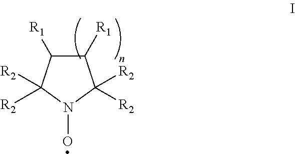

or an enantiomer, diastereomer, racemate, or pharmaceutically acceptable salt or solvate thereof,

wherein

R₁ each independently is selected from H, —OH, —COR₃, —COOR₃, —OCOOR₃, —OCON(R₃)₂, —(C₁-C₁₆)alkylene-COOR₃, —CN, —NO₂, —SH, —SR₃, —(C₁-C₁₆)alkyl, —O—(C₁-C₁₆)alkyl, —N(R₃)₂, —CON(R₃)₂, —SO₂R₃, —S(═O)R₃, or an NO-donor group of the formula —X₁-X₂-X₃, wherein X₁ is absent or selected from —O—, —S— or —NH—; X₂ is absent or is (C₁-C₂₀)alkylene optionally substituted by one or more —ONO₂ groups and optionally further substituted by a moiety of the general formula D:

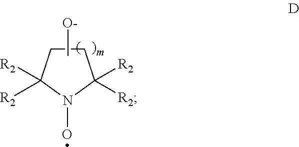

and X₃ is —NO or —ONO₂, provided that at least one R₁ group is an NO-donor group;

R₂ each independently is selected from (C₁-C₁₆)alkyl, (C₂-C₁₆)alkenyl, or (C₂-C₁₆)alkynyl;

R₃ each independently is selected from H, (C₁-C₈)alkyl, (C₃-C₁₀)cycloalkyl, 4-12-membered heterocyclyl, or (C₆-C₁₄)aryl, each of which other than H may optionally be substituted with —OH, —COR₄, —COOR₄, —OCOOR₄, —OCON(R₄)₂, —(C₁-C₈)alkylene-COOR₄, —CN, —NO₂, —SH, —SR₄, —(C₁-C₈)alkyl, —O—(C₁-C₈)alkyl, —N(R₄)₂, —CON(R₄)₂, —SO₂R₄, or —S(═O)R₄;

R₄ each independently is selected from H, (C₁-C₈)alkyl, (C₃-C₁₀)cycloalkyl, 4-12-membered heterocyclyl, or (C₆-C₁₄)aryl; and

n and m each independently is an integer of 1 to 3.

In another aspect, the present invention provides a pharmaceutical composition for prevention, treatment or management of PH comprising a compound of the general formula I as defined above, or an enantiomer, diastereomer, racemate, or pharmaceutically acceptable salt or solvate thereof, and a pharmaceutically acceptable carrier.

In still another aspect, the present invention provides a compound of the general formula I as defined above, or an enantiomer, diastereomer, racemate, or pharmaceutically acceptable salt or solvate thereof, for use in prevention, treatment or management of PH.

In yet another aspect, the present invention relates to use of a compound of the general formula I as defined above, or an enantiomer, diastereomer, racemate, or pharmaceutically acceptable salt or solvate thereof, for the preparation of a pharmaceutical composition for prevention, treatment or management of PH.

Whereas the aforesaid 3-nitratomethyl-2,2,5,5-tetramethylpyrrolidinyloxy is soluble in ethyl acetate and dimethylsulfoxide (DMSO), it is insoluble in non-toxic aqueous liquids that are suitable for human administration, such as water, saline, dextrose solution, and polyethylene glycol. It has further been found, in accordance with the present invention, that formulation of 1-pyrrolidinyloxy derivatives such as 3-nitratomethyl-2,2,5,5-tetramethylpyrrolidinyloxy into nanoparticulate particles produces a stable translucent suspension of up to 2 mg of the active agent/ml in water, saline, or dextrose solution. Such a suspension is readily sterile-filtered via a 0.22-μ filter and is well tolerated when injected parenterally into rodents.

In a further aspect, the present invention thus provides a water dispersible powder comprising nanoparticles comprising a compound of the general formula I as defined above, or an enantiomer, diastereomer, racemate, or pharmaceutically acceptable salt or solvate thereof.

In still a further aspect, the present invention provides a pharmaceutical composition comprising a water dispersible powder as defined above and a pharmaceutically acceptable carrier or diluent.

In yet a further aspect, the present invention relates to a method of prevention, treatment or management of PH in an individual in need thereof, comprising administering to said individual a pharmaceutical composition comprising a water dispersible powder as defined above and a pharmaceutically acceptable carrier or diluent.

### BRIEF DESCRIPTION OF DRAWINGS

FIG. 1 shows the effect of monocrotaline (MCT; 60 mg/kg) on mean pulmonary arterial pressure (MPAP), and the effect of compound 1a (R100; administered in an amount of 1.5 mg/kg/day BID, IP; or 2 mg/kg/day BID, PO, starting 38 days after MCT administration and during 10 days) on MCT-induced changes in MPAP.

FIGS. 2A-2C show effect of compound 1a on MCT-induced pulmonary vascular remodeling. Pulmonary vascular remodeling in rats treated with MCT+vehicle, PO (2A); Pulmonary vascular remodeling in rats treated with MCT+compound 1a (1.5 mg/kg/day BID, IP) (2B); Pulmonary vascular remodeling in rats treated with MCT+compound 1a (2 mg/kg/day BID, PO) (2C) (in each one of the figures, the top 3 panels are 10× and the bottom panel is 30×).

FIGS. 3A-3B show graphs demonstrating particle size (diameter, nanometers) distribution by number of the powder prepared when dispersed in water (3A) and in isotonic dextrose solution (3B).

### DETAILED DESCRIPTION OF THE INVENTION

In one aspect, the present invention provides a method for prevention, treatment or management of pulmonary hypertension (PH) by administration of piperidine, pyrrolidine, or azepane derivatives of the general formula I as defined above, comprising one to four NO donor groups and a reactive oxygen species (ROS) degradation catalyst, i.e., a superoxide scavenger.

The term “alkyl” as used herein typically means a straight or branched saturated hydrocarbon radical having 1-16 carbon atoms and includes, e.g., methyl, ethyl, n-propyl, isopropyl, n-butyl, sec-butyl, isobutyl, tert-butyl, n-pentyl, 2,2-dimethylpropyl, n-hexyl, n-heptyl, n-octyl, n-nonyl, n-decyl, n-undecyl, n-dodecyl, n-tridecyl, n-tetradecyl, n-pentadecyl, n-hexadecyl, and the like. Preferred are (C₁-C₆)alkyl groups, more preferably (C₁-C₄)alkyl groups, most preferably methyl and ethyl. The terms “alkenyl” and “alkynyl” typically mean straight and branched hydrocarbon radicals having 2-16 carbon atoms and 1 double or triple bond, respectively, and include ethenyl, propenyl, 3-buten-1-yl, 2-ethenylbutyl, 3-octen-1-yl, 3-nonenyl, 3-decenyl, and the like, and propynyl, 2-butyn-1-yl, 3-pentyn-1-yl, 3-hexynyl, 3-octynyl, 4-decynyl, and the like. C₂-C₆ alkenyl and alkynyl radicals are preferred, more preferably C₂-C₄ alkenyl and alkynyl.

The term “alkylene” typically means a divalent straight or branched hydrocarbon radical having 1-20 carbon atoms and includes, e.g., methylene, ethylene, propylene, butylene, 2-methylpropylene, pentylene, 2-methylbutylene, hexylene, 2-methylpentylene, 3-methylpentylene, 2,3-dimethylbutylene, heptylene, octylene and the like. Preferred are (C₁-C₈)alkylene, more preferably (C₁-C₄)alkylene, most preferably (C₁-C₂)alkylene.

The term “cycloalkyl” as used herein means a cyclic or bicyclic hydrocarbyl group having 3-12 carbon atoms such as cyclopropyl, cyclobutyl, cyclopentyl, cyclohexyl, cycloheptyl, cyclooctyl, adamantyl, bicyclo[3.2.1]octyl, bicyclo[2.2.1]heptyl, and the like. Preferred are (C₅-C₁₀)cycloalkyls, more preferably (C₅-C₇)cycloalkyls.

The term “aryl” denotes an aromatic carbocyclic group having 6-14 carbon atoms consisting of a single ring or multiple rings either condensed or linked by a covalent bond such as, but not limited to, phenyl, naphthyl, phenanthryl, and biphenyl.

The term “heterocyclic ring” denotes a mono- or poly-cyclic non-aromatic ring of 4-12 atoms containing at least one carbon atom and one to three heteroatoms selected from sulfur, oxygen or nitrogen, which may be saturated or unsaturated, i.e., containing at least one unsaturated bond. Preferred are 5- or 6-membered heterocyclic rings. The term “heterocyclyl” as used herein refers to any univalent radical derived from a heterocyclic ring as defined herein by removal of hydrogen from any ring atom. Examples of such radicals include, without limitation, piperidino, 4-morpholinyl, or pyrrolidinyl.

The term “NO-donor group” as defined herein refers to any group of the formula —X₁-X₂-X₃, wherein X₁ may be absent or is selected from —O—, —S— or —NH—; X₂ may be absent or is (C₁-C₂₀)alkylene optionally substituted by one or more —ONO₂ groups and optionally further substituted by a moiety of the general formula D as defined above; and X₃ is —NO or —ONO₂. Preferred NO-donor groups are those in which X₁ is absent or is —O—; X₂ is absent or is —(C₁-C₆)alkylene, preferably —(C₁-C₄)alkylene, more preferably methylene; and X₃ is —NO or —ONO₂, preferably —ONO₂, and said alkylene is optionally substituted as defined hereinabove. According to the method of the present invention, the compound of the general formula I may comprise one NO-donor group or more than one identical or different NO-donor groups.

In certain embodiments, the compound used according to the method of the present invention is a compound of the general formula I, wherein R₁ each independently is selected from H, —COOR₃, —CON(R₃)₂, or an NO-donor group; and R₃ is H.

In certain embodiments, the compound used according to the method of the present invention is a compound of the general formula I, wherein R₂ each independently is (C₁-C₈)alkyl, preferably (C₁-C₄)alkyl, more preferably (C₁-C₂)alkyl, most preferably methyl. Preferred embodiments are those in which all the R₂ groups in the formula I are identical.

In certain embodiments, the compound used according to the method of the present invention is a compound of the general formula I, wherein in said NO-donor group, X₁ is absent or —O—; X₂ is absent or (C₁-C₂₀)alkylene, preferably —(C₁-C₆)alkylene, more preferably —(C₁-C₄)alkylene, most preferably methylene; X₃ is —NO or —ONO₂, preferably —ONO₂; and said alkylene is optionally substituted by one or more —ONO₂ groups and optionally further substituted by a moiety of the general formula D as defined above.

In certain embodiments, the compound used according to the method of the present invention is a compound of the general formula I, wherein n is 1, 2 or 3, preferably 1 or 2.

In certain embodiments, the compound used according to the method of the present invention has the general formula I, wherein n is 1, i.e., a 1-pyrrolidinyloxy derivative of the formula Ia (see Table 1). In particular embodiments, the compound used according to this method has the general formula Ia, wherein either the carbon atom at position 3 of the pyrrolidine ring or the carbon atom at position 4 of the pyrrolidine ring, or both, are each linked to an NO-donor group.

In other certain embodiments, the compound used according to the method of the present invention has the general formula I, wherein n is 2, i.e., a 1-piperidinyloxy derivative of the formula Ib (see Table 1). In particular embodiments, the compound used according to this method has the general formula Ib, wherein one, two or three of the carbon atoms at positions 3 to 5 of the piperidine ring are each linked to an NO-donor group. In more particular embodiments, (i) the carbon atom at position 3 of the piperidine ring and optionally one or more of the carbon atoms at positions 4 or 5 of the piperidine ring are each linked to an NO-donor group; (ii) the carbon atom at position 4 of the piperidine ring and optionally one or more of the carbon atoms at positions 3 or 5 of the piperidine ring are each linked to an NO-donor group; or (iii) the carbon atom at position 5 of the piperidine ring and optionally one or more of the carbon atoms at positions 3 or 4 of the piperidine ring are each linked to an NO-donor group.

In further certain embodiments, the compound used according to the method of the present invention has the general formula I, wherein n is 3, i.e., a 1-azepanyloxy derivative of the formula Ic (see Table 1). In particular embodiments, the compound used according to this method has the general formula Ic, wherein one, two, three or four of the carbon atoms at positions 3 to 6 of the azepane ring are each linked to an NO-donor group. In more particular embodiments, (i) the carbon atom at position 3 of the azepane ring and optionally one or more of the carbon atoms at positions 4 to 6 of the azepane ring are each linked to an NO-donor group; (ii) the carbon atom at position 4 of the azepane ring and optionally one or more of the carbon atoms at positions 3, 5 or 6 of the azepane ring are each linked to an NO-donor group; (iii) the carbon atom at position 5 of the azepane ring and optionally one or more of the carbon atoms at positions 3, 4 or 6 of the azepane ring are each linked to an NO-donor group; or (iv) the carbon atom at position 6 of the azepane ring and optionally one or more of the carbon atoms at positions 3 to 5 of the azepane ring are each linked to an NO-donor group.

In particular embodiments, the compound used according to the method of the invention is a 1-pyrrolidinyloxy derivative of the formula Ia, 1-piperidinyloxy derivative of the formula Ib, or 1-azepanyloxy derivative of the formula Ic, and each one of the NO-donor groups in said compound independently is of the formula —(C₁-C₆)alkylene-ONO₂, preferably —(C₁-C₄)alkylene-ONO₂, more preferably —CH₂—ONO₂, or —O—(C₁-C₆)alkylene-ONO₂, wherein said alkylene is optionally substituted by one or more —ONO₂ groups, or is —ONO₂.

Specific compounds of the general formulas Ia, Ib and Ic described herein, in which each one of the R₁ groups independently is either H or the NO-donor group —CH₂—ONO₂ or —ONO₂, are herein identified compounds 1a/1b-15a/1b in bold (compound 1a is also identified R100), and their full chemical structures are depicted in Table 2. Other specific compounds of the general formulas Ia and Ib described herein, in which one R₁ group is the NO-donor group —CH₂—ONO₂ or —ONO₂, and another R₁ group is not H, are herein identified compounds 16a/1b-17a/1b in bold, and their full chemical structures are depicted in Table 3. A further specific compound of the general formula Ib described herein, in which one R₁ group is the NO-donor group —O—CH₂—CH(ONO₂)CH₂—ONO₂, and the other R₁ groups are H, is herein identified compound 18 in bold, and its full chemical structure is depicted in Table 3.

TABLE 1
Structures Ia, Ib and Ic, indicating 1-pyrrolidinyloxy, 1-piperidinyloxy
and 1-azepanyloxy derivatives, respectively

| Ia | Ib | Ic |
|---|---|---|
| 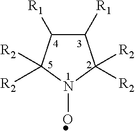 | 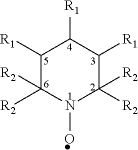 | 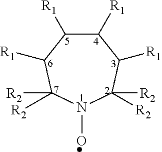 |

In specific embodiments, the compound used according to the method of the invention is the compound of formula Ia, i.e., a compound of the general formula I in which n is 1, wherein R₂ each is methyl; and (i) the R₁ group linked to the carbon atom at position 3 of the pyrrolidine ring is the NO-donor group —CH₂—ONO₂ or ONO₂; and the R₁ group linked to the carbon atom at position 4 of the pyrrolidine ring is H, i.e., 3-nitratomethyl-2,2,5,5-tetramethylpyrrolidinyloxy (compound 1a; R100) or 3-nitrato-2,2,5,5-tetramethylpyrrolidinyloxy (compound 1b), respectively; or (ii) each one of the R₁ groups linked to the carbon atoms at positions 3 and 4 of the pyrrolidine ring is the NO-donor group —CH₂—ONO₂ or ONO₂, i.e., 3,4-dinitrato methyl-2,2,5,5-tetramethylpyrrolidinyloxy (compound 2a) or 3,4-dinitrato-2,2,5,5-tetramethylpyrrolidinyloxy (compound 2b), respectively.

In other specific embodiments, the compound used according to the method of the invention is the compound of formula Ib, i.e., a compound of the general formula I wherein n is 2, wherein R₂ each is methyl; and (i) the R₁ group linked to the carbon atom at position 3 of the piperidine ring is the NO-donor group —CH₂—ONO₂ or ONO₂; and each one of the R₁ groups linked to the carbon atoms at positions 4 and 5 of the piperidine ring is H, i.e., 3-nitratomethyl-2,2,6,6-tetramethylpiperidinyloxy (3-nitratomethyl-TEMPO; compound 3a) or 3-nitrato-2,2,6,6-tetramethylpiperidinyloxy (3-nitrato-TEMPO; compound 3b), respectively; (ii) the R₁ group linked to the carbon atom at position 4 of the piperidine ring is the NO-donor group —CH₂—ONO₂ or ONO₂; and each one of the R₁ groups linked to the carbon atoms at positions 3 and 5 of the piperidine ring is H, i.e., 4-nitratomethyl-2,2,6,6-tetramethylpiperidinyloxy (4-nitratomethyl-TEMPO; compound 4a) or 4-nitrato-2,2,6,6-tetramethylpiperidinyloxy (3-nitrato-TEMPO; compound 4b), respectively; (iii) each one of the R₁ groups linked to the carbon atoms at positions 3 and 4 of the piperidine ring is the NO-donor group —CH₂—ONO₂ or ONO₂; and the R₁ group linked to the carbon atom at position 5 of the piperidine ring is H, i.e., 3,4-dinitratomethyl-2,2,6,6-tetramethylpiperidinyloxy (3,4-dinitratomethyl-TEMPO; compound 5a) or 3,4-dinitrato-2,2,6,6-tetramethylpiperidinyloxy (3,4-dinitrato-TEMPO; compound 5b), respectively; (iv) each one of the R₁ groups linked to the carbon atoms at positions 3 and 5 of the piperidine ring is the NO-donor group —CH₂—ONO₂ or ONO₂; and the R₁ group linked to the carbon atom at position 4 of the piperidine ring is H, i.e., 3,5-dinitratomethyl-2,2,6,6-tetramethylpiperidinyloxy (3,5-dinitratomethyl-TEMPO; compound 6a) or 3,5-dinitrato-2,2,6,6-tetramethyl piperidinyloxy (3,5-dinitrato-TEMPO; compound 6b), respectively; or (v) each one of the R₁ groups linked to the carbon atoms at positions 3 to 5 of the piperidine ring is the NO-donor group —CH₂—ONO₂ or ONO₂, i.e., 3,4,5-trinitratomethyl-2,2,6,6-tetramethylpiperidinyloxy (3,4,5-trinitratomethyl-TEMPO; compound 7a) or 3,4,5-trinitrato-2,2,6,6-tetramethylpiperidinyloxy (3,4,5-trinitrato-TEMPO; compound 7b), respectively.

In further specific embodiments, the compound used according to the method of the invention is the compound of formula Ic, i.e., a compound of the general formula I wherein n is 3, wherein R₂ each is methyl; and (i) the R₁ group linked to the carbon atom at position 3 of the azepane ring is the NO-donor group —CH₂—ONO₂ or ONO₂; and each one of the R₁ groups linked to the carbon atoms at positions 4 to 6 of the azepane ring is H, i.e., 3-nitratomethyl-2,2,7,7-tetramethylazepanyloxy (compound 8a) or 3-nitrato-2,2,7,7-tetramethylazepanyloxy (compound 8b), respectively; (ii) the R₁ group linked to the carbon atom at position 4 of the azepane ring is the NO-donor group —CH₂—ONO₂ or ONO₂; and each one of the R₁ groups linked to the carbon atoms at position 3, 5 and 6 of the azepane ring is H, i.e., 4-nitratomethyl-2,2,7,7-tetramethylazepanyloxy (compound 9a) or 4-nitrato-2,2,7,7-tetramethylazepanyloxy (compound 9b), respectively; (iii) each one of the R₁ groups linked to the carbon atoms at positions 3 and 4 of the azepane ring is the NO-donor group —CH₂—ONO₂ or ONO₂; and each one of the R₁ groups linked to the carbon atoms at positions 5 and 6 of the azepane ring is H, i.e., 3,4-dinitratomethyl-2,2,7,7-tetramethylazepanyloxy (compound 10a) or 3,4-dinitrato-2,2,7,7-tetra methylazepanyloxy (compound 10b), respectively; (iv) each one of the R₁ groups linked to the carbon atoms at positions 3 and 5 of the azepane ring is the NO-donor group —CH₂—ONO₂ or ONO₂; and each one of the R₁ groups linked to the carbon atoms at positions 4 and 6 of the azepane ring is H, i.e., 3,5-dinitratomethyl-2,2,7,7-tetramethylazepanyloxy (compound 11a) or 3,5-dinitrato-2,2,7,7-tetramethyl azepanyloxy (compound 11b), respectively; (v) each one of the R₁ groups linked to the carbon atoms at positions 3 and 6 of the azepane ring is the NO-donor group —CH₂—ONO₂ or ONO₂; and each one of the R₁ groups linked to the carbon atoms at positions 4 and 5 of the azepane ring is H, i.e., 3,6-dinitratomethyl-2,2,7,7-tetramethylazepanyloxy (compound 12a) or 3,6-dinitrato-2,2,7,7-tetramethyl azepanyloxy (compound 12b), respectively; (vi) each one of the R₁ groups linked to the carbon atoms at positions 3 to 5 of the azepane ring is the NO-donor group —CH₂—ONO₂ or ONO₂; and the R₁ group linked to the carbon atom at position 6 of the azepane ring is H, i.e., 3,4,5-trinitratomethyl-2,2,7,7-tetramethylazepanyloxy (compound 13a) or 3,4,5-trinitrato-2,2,7,7-tetramethylazepanyloxy (compound 13b), respectively; (vii) each of the R₁ groups linked to the carbon atoms at positions 3, 4 and 6 of the azepane ring is the NO-donor group —CH₂—ONO₂ or ONO₂; and the R₁ group linked to the carbon atom at position 5 of the azepane ring is H, i.e., 3,4,6-trinitratomethyl-2,2,7,7-tetramethylazepanyloxy (compound 14a) or 3,4,6-trinitrato-2,2,7,7-tetramethylazepanyloxy (compound 14b), respectively; or (viii) each of the R₁ groups linked to the carbon atoms at positions 3 to 6 of the azepane ring is the NO-donor group —CH₂—ONO₂ or ONO₂, i.e., 3,4,5,6-tetranitratomethyl-2,2,7,7-tetramethylazepanyloxy (compound 15a) or 3,4,5,6-tetranitrato-2,2,7,7-tetramethylazepanyloxy (compound 15b), respectively.

In still other specific embodiments, the compound used according to the method of the invention is the compound of formula Ia, wherein R₂ each is methyl; the R₁ group linked to the carbon atom at position 3 of the pyrrolidine ring is the NO-donor group —CH₂—ONO₂ or —ONO₂; and the R₁ group linked to the carbon atom at position 4 of the pyrrolidine ring is —CONH₂, i.e., 3-nitratomethyl-4-carbamoyl-2,2,5,5-tetramethylpyrrolidinyloxy (compound 16a) or 3-nitrato-4-carbamoyl-2,2,5,5-tetramethylpyrrolidinyloxy (compound 16b), respectively.

In yet other specific embodiments, the compound used according to the method of the invention is the compound of formula Ib, wherein R₂ each is methyl; the R₁ group linked to the carbon atom at position 3 of the piperidine ring is the NO-donor group —CH₂—ONO₂ or —ONO₂; the R₁ group linked to the carbon atom at position 4 of the piperidine ring is —COOH; and the R₁ group linked to the carbon atoms at position 5 of the piperidine ring is H, i.e., 3-nitratomethyl-4-carboxy-2,2,6,6-tetramethylpiperidinyloxy (3-nitratomethyl-4-carboxy-TEMPO; compound 17a) or 3-nitrato-4-carboxy-2,2,6,6-tetramethylpiperidinyloxy (3-nitrato-4-carboxy-TEMPO; compound 17b), respectively.

In still a further specific embodiment, the compound used according to the method of the invention is the compound of formula Ib, wherein R₂ each is methyl; the R₁ group linked to the carbon atom at position 4 of the piperidine ring is the NO-donor group —O—CH₂—CH(ONO₂)CH₂—ONO₂; and each one of the R₁ groups linked to the carbon atom at position 3 and 5 of the piperidine ring is H, i.e., 4-(2,3-dinitratopropyloxy)-2,2,6,6-tetramethylpiperidinyloxy (4-(2,3-dinitratopropyloxy)-TEMPO; compound 18).

TABLE 2
Compounds of the general formulas Ia, Ib and Ic, identified 1a-15a*

| | | |
|---|---|---|
| 1a | 2a | 3a |
| 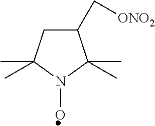 | 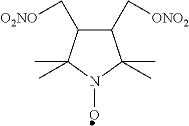 | 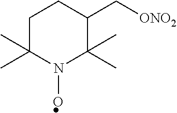 |
| 4a | 5a | 6a |
| 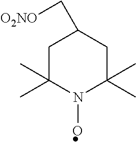 | 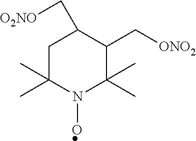 | 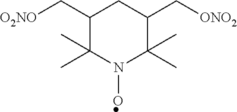 |
| 7a | 8a | 9a |
| 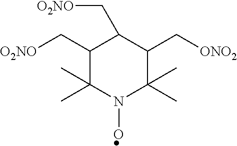 | 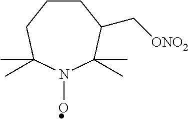 | 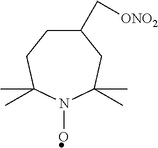 |
| 10a | 11a | 12a |
| 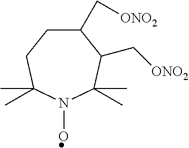 | 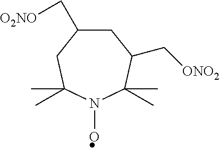 | 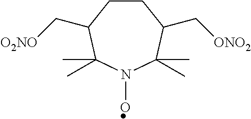 |
| 13a | 14a | 15a |
| 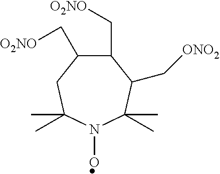 | 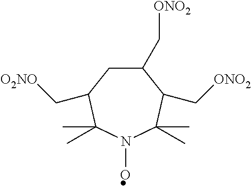 | 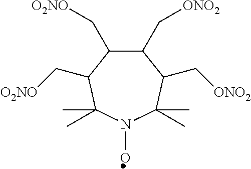 |

*The compounds corresponding to 1a-15a, in which each one of the —CH₂—ONO₂ groups is replaced by the —ONO₂ group, are herein identified compounds 1b-15b

TABLE 3
Compounds of the general formulas Ia and Ib, identified 16a-17a* and 18

| 16a | 17a | 18 |
|---|---|---|
| 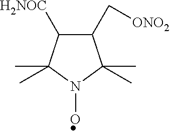 | 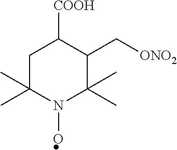 | 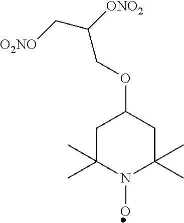 |

*The compounds corresponding to 16a and 17a, in which each one of the —CH₂—ONO₂ groups is replaced by the —ONO₂ group, are herein identified compounds 16b and 17b

In other particular embodiments, the compound used according to the method of the present invention is a 1-pyrrolidinyloxy derivative of the formula Ia, 1-piperidinyloxy derivative of the formula Ib, or 1-azepanyloxy derivative of the formula Ic; wherein at least one of the NO-donor groups in said compound is of the formula —O—(C₁-C₆)alkylene-ONO₂; and said alkylene is substituted by a moiety of the general formula D as defined above, and is optionally further substituted by one or more —ONO₂ groups. The general formula D, in which oxygen atom is linked to the carbon atom at position 3 or 4 of the ring, represents a 3-hydroxy-pyrrolidinoxy, 3- or 4-hydroxy-piperidinyloxy, or 3- or 4-hydroxy-azepanyloxy derivative. Conceptually, the compound used in this case is thus a dimer- or higher multimer-like compound, in which two or more identical or different entities, each independently being selected from 1-pyrrolidinyloxy, 1-piperidinyloxy or 1-azepanyloxy derivatives, are linked via alkylene bridges substituted by one or more —ONO₂ groups, wherein each alkylene bridge links two entities only.

Preferred dimer- or higher multimer-like compounds to be used according to the method of the invention are those in which (i) a 1-pyrrolidinyloxy derivative of the general formula Ia is linked via one or two NO-donor groups thereof to one or two identical or different moieties of a 3-hydroxy-pyrrolidinoxy, i.e., one or two moieties of the general formula D in which m is 1; (ii) a 1-piperidinyloxy derivative of the general formula Ib is linked via one, two or three NO-donor groups thereof to one, two or three identical or different moieties of a 3-, or 4-hydroxy-piperidinyloxy, i.e., one to three moieties of the general formula D in which m is 2; or (iii) a 1-azepanyloxy derivative of the general formula Ic is linked via one, two, three or four NO-donor groups thereof to one, two, three or four identical or different moieties of a 3-, or 4-hydroxy-azepanyloxy, i.e., one to four moieties of the general formula D in which m is 3.

Specific compounds of the general formula Ib described herein, having a dimer-like structure, are herein identified compounds 19-20 in bold, and their full chemical structures are depicted in Table 4.

In specific embodiments, the compound used according to the method of the invention is the compound of formula Ib, wherein each one of R₁ linked to the carbon atoms at positions 3 and 5 of the piperidine ring is H; and (i) R₁ linked to the carbon atom at position 4 of the piperidine ring is the NO-donor group —O—CH₂—CH(ONO₂)—CH(ONO₂)—CH₂-D, wherein in the general formula D, m is 2, and the oxygen atom is linked to the carbon atom at position 4 of the piperidine ring; and R₂ each is methyl, i.e., 1,4-di-(4-oxo-TEMPO)-2,3-dinitratobutane (compound 19); or (ii) R₁ linked to the carbon atom at position 4 of the piperidine ring is the NO-donor group —O—CH₂—CH(ONO₂)—CH₂-D, wherein in the general formula D, m is 2, and the oxygen atom is linked to the carbon atom at position 4 of the piperidine ring; and R₂ each is methyl, i.e., 1,3-di-(4-oxo-TEMPO)-2-nitratopropane (compound 20).

TABLE 4
Compounds of the general formula Ib, identified 19-20

| 19 | 20 |
|---|---|
| 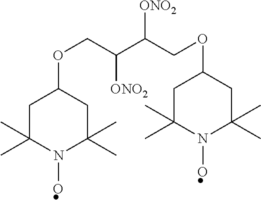 | 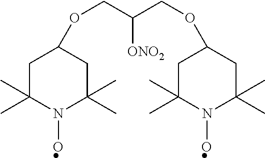 |

The compounds of the general formula I may be synthesized according to any technology or procedure known in the art, e.g., as described in detail in U.S. Pat. Nos. 6,448,267, 6,455,542 and 6,759,430.

The compounds of the general formula I may have one or more asymmetric centers, and may accordingly exist both as enantiomers, i.e., optical isomers (R, S, or racemate, wherein a certain enantiomer may have an optical purity of 90%, 95%, 99% or more) and as diastereoisomers. Specifically, those chiral centers may be, e.g., in each one of the carbon atoms of the 1-pyrrolidinyloxy derivative, 1-piperidinyloxy derivative; and 1-azepanyloxy derivative of the general formulas Ia, Ib and Ic, respectively. According to the method of the present invention, prevention, treatment or management of pulmonary hypertension can be carried out by administration of all such enantiomers, isomers and mixtures thereof, as well as pharmaceutically acceptable salts and solvates thereof.

Optically active forms of the compounds of the general formula I may be prepared using any method known in the art, e.g., by resolution of the racemic form by recrystallization techniques; by chiral synthesis; by extraction with chiral solvents; or by chromatographic separation using a chiral stationary phase. A non-limiting example of a method for obtaining optically active materials is transport across chiral membranes, i.e., a technique whereby a racemate is placed in contact with a thin membrane barrier, the concentration or pressure differential causes preferential transport across the membrane barrier, and separation occurs as a result of the non-racemic chiral nature of the membrane that allows only one enantiomer of the racemate to pass through. Chiral chromatography, including simulated moving bed chromatography, can also be used. A wide variety of chiral stationary phases are commercially available.

The term “pulmonary hypertension” (PH) as used herein refers to a severe disease characterized by increased pulmonary vascular resistance, pulmonary arterial pressure (PAP), and ultimately pulmonary vascular remodeling effects that interfere with ventilation-perfusion relationships and compromise ventricular function. Several classification systems for PH have been published, including the Evian Nomenclature and Classification of PH (1998) and the Revised Nomenclature and Classification of PH (2003) (McCrory and Lewis, 2004).

PH may be either primary or secondary, and as stated above, is currently classified into five groups, wherein pulmonary arterial hypertension (PAH) is classified as Group 1; PH associated with left heart diseases is classified as Group 2; PH associated with lung diseases and/or hypoxemia is classified as Group 3; PH due to chronic thrombotic and/or embolic diseases is classified as Group 4; and PH of other origin is classified as Group 5 (Galiè et al., 2004).

The term PAH as used herein refers to any PAH including, without being limited to, idiopathic PAH (IPAH); familial PAH (FPAH); PAH associated with collagen vascular disease, e.g., scleroderma; PAH associated with congenital heart disorders, e.g., congenital shunts between the systemic and pulmonary circulation, portal hypertension; PAH associated with HIV infection; PAH associated with venous or capillary diseases; PAH associated with thyroid disorders, glycogen storage disease, Gaucher's disease, hemoglobinopathies, or myeloproliferative disorders; PAH associated with either smoke inhalation or combined smoke inhalation and burn injury; PAH associated with aspiration; PAH associated with ventilator injury; PAH associated with pneumonia; PAH associated with Adult Respiratory Distress Syndrome; persistent PH of the newborn; neonatal respiratory distress syndrome of prematurity; neonatal meconium aspiration; neonatal diaphragmatic hernia; pulmonary capillary hemangiomatosis; and pulmonary veno-occlusive disease.

Examples of left heart disease that may be associated with Group 2 PH include, without limiting, left sided atrial or ventricular diseases, and valvular diseases, e.g., mitral stenosis.

Examples of lung diseases that may be associated with Group 3 PH include, without being limited to, chronic obstructive pulmonary disease (COPD), interstitial lung diseases (ILD), sleep-disordered breathing, alveolar hypoventilation disorders, chronic exposure to high altitude, and developmental lung abnormalities.

Examples of chronic thrombotic and/or embolic diseases that may be associated with Group 4 PH include, without limiting, thromboembolic obstruction of distal or proximal pulmonary arteries, and non-thrombotic pulmonary embolism of, e.g., tumor cells or parasites.

Examples of disorders or diseases that may be associated with Group 5 PH include, without being limited to, compression of pulmonary vessels by adenopathy, fibrosing mediastinitis, lymphangiomatosis, pulmonary Langerhans' cell granulomatosis (histiocytosis), sarcoidosis, hemoglobinopathy, and tumors.

Many of the diseases, disorders and conditions listed above can be associated with increased risk for PH, wherein particular examples, without limiting, include congenital heart disease, e.g., Eisenmenger syndrome; left heart disease; pulmonary venous disease, e.g., fibrosis tissue narrowing or occluding pulmonary veins and venules; pulmonary arterial disease; diseases causing alveolar hypoxia; fibrotic lung diseases; Williams syndrome; subjects with intravenous drug abuse injury; pulmonary vasculitis such as Wegener's, Goodpasture's, and Churg-Strauss syndromes; emphysema; chronic bronchitis; kyphoscoliosis; cystic fibrosis; obesity-hyper-ventilation and sleep apnea disorders; pulmonary fibrosis; sarcoidosis; silocosis; CREST (calcinosis cutis, Raynaud phenomenon; esophageal motility disorder; sclerodactyl), and teleangiectasia) and other connective tissue diseases. For example, a subject who possesses a bone morphogenetic protein receptor E (BMPR2) mutation has a 10-20% lifetime risk of acquiring FPAH, and subjects with hereditary hemorrhagic telangiectasa, particularly those carrying mutations in ALK1, were also identified as being at risk for IPAH. Risk factors and diagnostic criteria for PH are described in McGoon et al., 2004.

The method of the present invention can be used for treatment any form of PH including, but not limited to, mild, i.e., associated with an increase of up to 30, more particularly 20-30, mmHg in mean pulmonary arterial pressure (MPAP) at rest; moderate, i.e., associated with an increase of 30-39 mmHg in MPAP at rest; and severe, i.e., associated with an increase of 40 mmHg or more in MPAP at rest.

The term “treatment” as used herein with respect to PH refers to administration of a compound of the general formula I as defined above, or an enantiomer, diastereomer, racemate, or pharmaceutically acceptable salt or solvate thereof, after the onset of symptoms of PH in any of its forms. The term “prevention” as used herein with respect to PH refers to administration of said compound prior to the onset of symptoms, particularly to patients at risk for PH; and the term “management” as used herein with respect to PH refers to prevention of recurrence of PH in a patient previously suffered from PH. The term “therapeutically effective amount” as used herein refers to the quantity of the compound of the general formula I as defined above, or an enantiomer, diastereomer, racemate, or pharmaceutically acceptable salt or solvate thereof, that is useful to treat, prevent or manage the PH.

As shown in the Examples section hereinafter, administration of compound 1a in a rat PH model, starting 38 days after monocrotaline (MCT; a plant poison that induces a well-characterized experimental model of PH) administration and during 10 days, significantly reduced the elevation of PAH developed following MCT administration. Furthermore, chronic treatment with compound 1a remarkably reduced the alveolar damage, the inflammatory cell infiltrate, and the vascular smooth muscle hypertrophy as compared to the vehicle control. These findings are of high significance in view of the fact that the onset of compound 1a therapy was delayed after MCT injection and begun at a timepoint of established PAH and lung injury.

In another aspect, the present invention provides a pharmaceutical composition for prevention, treatment or management of PH comprising a compound of the general formula I as defined above, or an enantiomer, diastereomer, racemate, or pharmaceutically acceptable salt or solvate thereof, herein also identified “the active agent”, and a pharmaceutically acceptable carrier. In particular embodiments, the pharmaceutical composition of the invention comprises a compound selected from compounds 1a, 1b, 2a, 2b, 3a, 3b, 4a, 4b, 5a, 5b, 6a, 6b, 7a, 7b, 8a, 8b, 9a, 9b, 10a, 10b, 11a, 11b, 12a, 12b, 13a, 13b, 14a, 14b, 15a, 15b, 16a, 16b, 17a, 17b, 18, 19 or 20, preferably compound 1a, or an enantiomer, diastereomer, racemate, or pharmaceutically acceptable salt or solvate thereof.

The pharmaceutical compositions of the present invention can be provided in a variety of formulations, e.g., in a pharmaceutically acceptable form and/or in a salt form, as well as in a variety of dosages.

In one embodiment, the pharmaceutical composition of the present invention comprises a non-toxic pharmaceutically acceptable salt of the active agent. Suitable pharmaceutically acceptable salts include acid addition salts such as, without being limited to, those formed with hydrochloric acid, fumaric acid, p-toluenesulfonic acid, maleic acid, succinic acid, acetic acid, citric acid, tartaric acid, carbonic acid, or phosphoric acid. Salts of amine groups may also comprise quaternary ammonium salts in which the amino nitrogen atom carries a suitable organic group such as an alkyl, alkenyl, alkynyl, or aralkyl moiety. Furthermore, where the compounds of the general formula I carry an acidic moiety, suitable pharmaceutically acceptable salts thereof may include metal salts such as alkali metal salts, e.g., sodium or potassium salts, and alkaline earth metal salts, e.g., calcium or magnesium salts.

The pharmaceutically acceptable salts of the present invention may be formed by conventional means, e.g., by reacting the free base form of the active agent with one or more equivalents of the appropriate acid in a solvent or medium in which the salt is insoluble, or in a solvent such as water which is removed in vacuo or by freeze drying, or by exchanging the anions of an existing salt for another anion on a suitable ion exchange resin.

The present invention encompasses solvates of the active agent as well as salts thereof, e.g., hydrates.

The pharmaceutical compositions provided by the present invention may be prepared by conventional techniques, e.g., as described in Remington: The Science and Practice of Pharmacy, 19^{th} Ed., 1995. The compositions can be prepared, e.g., by uniformly and intimately bringing the active agent into association with a liquid carrier, a finely divided solid carrier, or both, and then, if necessary, shaping the product into the desired formulation. The compositions may be in liquid, solid or semisolid form and may further include pharmaceutically acceptable fillers, carriers, diluents or adjuvants, and other inert ingredients and excipients.

The compositions can be formulated for any suitable route of administration, but they are preferably formulated for parenteral administration, e.g., intravenous, intraarterial, intramuscular, subcutaneous or intraperitoneal administration, as well as for inhalation. The dosage will depend on the state of the patient, and will be determined as deemed appropriate by the practitioner. In particular embodiments, the dosage is 0.001-20 mg/kg, preferably 0.01-15 mg/kg, more preferably 0.1-10 mg/kg, still more preferably 0.1-5 mg/kg. The pharmaceutical compositions of the invention, particularly when used for treatment or prevention of PH, may be administered continuously, daily, twice daily, thrice daily or four times daily and/or upon the occurrence of symptoms associated with the condition, for various duration periods, e.g., weeks, months, years, or decades.

The pharmaceutical composition of the invention may be in the form of a sterile injectable aqueous or oleagenous suspension, which may be formulated according to the known art using suitable dispersing, wetting or suspending agents. The sterile injectable preparation may also be a sterile injectable solution or suspension in a non-toxic parenterally acceptable diluent or solvent. Acceptable vehicles and solvents that may be employed include, without limiting, water, Ringer's solution and isotonic sodium chloride solution.

Pharmaceutical compositions according to the present invention, when formulated for inhalation, may be administered utilizing any suitable device known in the art, such as metered dose inhalers, dry powder inhalers, liquid nebulizers, sprayers, thermal vaporizers, electrohydrodynamic aerosolizers, and the like. Particular inhalation methods and devices include, without limiting, those disclosed in U.S. Pat. Nos. 5,277,195, 5,320,094, 5,327,883, 5,364,838, 5,404,871, 5,419,315, 5,492,112, 5,506,203, 5,518,998, 5,558,085, 5,577,497, 5,622,166, 5,645,051, 5,654,007, 5,655,523, 5,658,878, 5,661,130, 5,672,581, 5,743,250, 5,780,014, 6,060,069, 6,238,647, 6,241,969, 6,335,316, 6,616,914 and 7,678,364; US Patent Publication No. 20020006901; and International Patent Publication Nos. WO95/24183, WO96/32149 and WO98/33480.

The abbreviations “MMAD” and “MMEAD” are well known in the art, and stand for the terms “mass median aerodynamic diameter” and “mass median equivalent aerodynamic diameter”, respectively, which are substantially equivalent.

The “aerodynamic equivalent” size of a particle is the diameter of a unit density sphere which exhibits the same aerodynamic behavior as the particle, regardless of actual density or shape. MMAD is usually determined using a cascade impactor, which measures the particle size as a function of the aerodynamic behavior of the particle in a high velocity air stream. The median particle size is obtained from a linear regression analysis of the cumulative distribution data. In one embodiment, the inhalation device delivers small particles, e.g., particles having MMAD of less than about 10 μm.

The inhalation device is preferably practical in the sense of being easy to use, small enough to carry conveniently, capable of providing multiple doses, and durable. Non-limiting examples of commercially available inhalation devices include Turbohaler (Astra, Wilmington, Del.), Rotahaler (Glaxo, Research Triangle Park, N.C.), Diskus (Glaxo, Research Triangle Park, N.C.), the Ultravent nebulizer (Mallinckrodt), the Acorn II nebulizer (Marquest Medical Products, Totowa, N.J.), and the Ventolin metered dose inhaler (Glaxo, Research Triangle Park, N.C.).

The formulation of the composition of the present invention, as well as the quantity of the formulation delivered and the duration of administration of a single dose, depend, inter alia, on the type of inhalation device employed. For some aerosol delivery systems such as nebulizers, the frequency of administration and duration of time for which the system is activated will mainly depend on the concentration of the active agent in the aerosol, wherein shorter periods of administration can be used with nebulizer solutions containing higher concentrations of the active agent. Devices such as metered dose inhalers can produce higher aerosol concentrations and can thus be operated for shorter periods to deliver the desired amount of the active agent. Devices such as dry powder inhalers deliver active agent until a given quantity of agent, determining the dose for a single administration, is expelled from the device. The formulation of the active agent is selected to yield the desired particle size in the chosen inhalation device.

Dry powder generation typically employs a method such as a scraper blade or an air blast to generate particles from a solid formulation of the active agent. The particles are generally generated in a container and then transported into the lung of a patient via a carrier air stream. Typically, in current dry powder inhalers, the force for breaking up the solid and airflow is provided solely by the patient's inhalation. One suitable dry powder inhaler is the Turbohaler (Astra, Wilmington, Del.).

Formulations of the active agent for administration from a dry powder inhaler typically include a finely divided dry powder containing said active agent as well as a bulking agent, buffer, carrier, and/or excipient. Additional additives can be added to the formulation, e.g., to dilute the powder as required for delivery from the particular powder inhaler; to facilitate processing of the formulation; to provide advantageous powder properties to the formulation; to facilitate dispersion of the powder from the inhalation device; to stabilize the formulation; and/or to provide taste to the formulation. Non-limiting examples of typical additives include mono-, di-, and polysaccharides; sugar alcohols and other polyols, such as lactose, glucose, raffinose, melezitose, lactitol, maltitol, trehalose, sucrose, mannitol, starch, or combinations thereof; and surfactants such as sorbitols, diphosphatidyl choline, or lecithin.

In some embodiments, a spray including the active agent can be produced by forcing a suspension or solution of the active agent through a nozzle under pressure. The nozzle size and configuration, the pressure applied, and the liquid feed rate can be chosen to achieve the desired output and particle size. An electrospray can be produced by an electric field in connection with a capillary or nozzle feed. Formulations suitable for use with a sprayer can include the active agent in an aqueous solution at a concentration of about 1-20 mg/ml. The formulation can include additional ingredients such as an excipient, buffer, isotonicity agent, preservative, surfactant, and/or zinc. The active agent can be administered by a nebulizer, such as a jet nebulizer, in which a compressed air source is used to create a high-velocity air jet through an orifice, or an ultrasonic nebulizer. Formulations of the active agent suitable for use with a nebulizer, either jet or ultrasonic, typically include the active agent in an aqueous solution, and optionally additional ingredients such as an excipient, buffer, isotonicity agent, preservative, surfactant, and/or zinc. The formulation can further include an excipient or ingredient for stabilization of the active agent such as a buffer, reducing agent, bulk protein, or carbohydrate. Bulk proteins, surfactants, carbohydrates and other additives are useful in formulating the active agent and can be used as described above.

In a metered dose inhaler, the active agent together with a propellant, an excipient and/or other additives are contained in a canister as a mixture including a liquefied compressed gas.

Pharmaceutical compositions according to the present invention, when formulated for administration route other than parenteral administration, may be in a form suitable for oral use, e.g., as tablets, troches, lozenges, aqueous, or oily suspensions, dispersible powders or granules, emulsions, hard or soft capsules, or syrups or elixirs. Compositions intended for oral use may be prepared according to any method known to the art for the manufacture of pharmaceutical compositions and may further comprise one or more agents selected from sweetening agents, flavoring agents, coloring agents and preserving agents in order to provide pharmaceutically elegant and palatable preparations. Tablets contain the active agent in admixture with non-toxic pharmaceutically acceptable excipients, which are suitable for the manufacture of tablets. These excipients may be, e.g., inert diluents such as calcium carbonate, sodium carbonate, lactose, calcium phosphate, or sodium phosphate; granulating and disintegrating agents, e.g., corn starch or alginic acid; binding agents, e.g., starch, gelatin or acacia; and lubricating agents, e.g., magnesium stearate, stearic acid, or talc. The tablets may be either uncoated or coated utilizing known techniques to delay disintegration and absorption in the gastrointestinal tract and thereby provide a sustained action over a longer period. For example, a time delay material such as glyceryl monostearate or glyceryl distearate may be employed. They may also be coated using the techniques described in the U.S. Pat. Nos. 4,256,108, 4,166,452 and 4,265,874 to form osmotic therapeutic tablets for control release. The pharmaceutical composition of the invention may also be in the form of oil-in-water emulsion.

The pharmaceutical compositions of the invention may be formulated for controlled release of the active agent. Such compositions may be formulated as controlled-release matrix, e.g., as controlled-release matrix tablets in which the release of a soluble active agent is controlled by having the active diffuse through a gel formed after the swelling of a hydrophilic polymer brought into contact with dissolving liquid (in vitro) or gastro-intestinal fluid (in vivo). Many polymers have been described as capable of forming such gel, e.g., derivatives of cellulose, in particular the cellulose ethers such as hydroxypropyl cellulose, hydroxymethyl cellulose, methylcellulose or methyl hydroxypropyl cellulose, and among the different commercial grades of these ethers are those showing fairly high viscosity. In other configurations, the compositions comprise the active agent formulated for controlled release in microencapsulated dosage form, in which small droplets of the active agent are surrounded by a coating or a membrane to form particles in the range of a few micrometers to a few millimeters.

Another contemplated formulation is depot systems, based on biodegradable polymers, wherein as the polymer degrades, the active agent is slowly released. The most common class of biodegradable polymers is the hydrolytically labile polyesters prepared from lactic acid, glycolic acid, or combinations thereof, e.g., poly(D,L-lactide) (PLA), poly(glycolide) (PGA), and the copolymer poly(D,L-lactide-co-glycolide) (PLG).

In still another aspect, the present invention provides a compound of the general formula I as defined above, or an enantiomer, diastereomer, racemate, or pharmaceutically acceptable salt or solvate thereof, for use in prevention, treatment or management of PH.

In yet another aspect, the present invention relates to use of a compound of the general formula I as defined above, or an enantiomer, diastereomer, racemate, or pharmaceutically acceptable salt or solvate thereof, for the preparation of a pharmaceutical composition for prevention, treatment or management of PH.

Poorly water-soluble drugs are often the keys for treatment of many diseases. Thus, it is an arduous task and challenge for scientists to generate a method for the formulation of such drugs so as to improve their solubility and bioavailability within the human body. Nanoparticles formulations comprising water-insoluble drugs have been disclosed, e.g., in US Patent Publication No. 2008/0187595, which discloses nanoparticles-containing compositions for transferring therapeutically active substances into cells, in particular cancer cells. International Patent Publication No. WO2009/126938 provides compositions comprising nanoparticles comprising a drug, e.g., a hydrophobic drug, and a carrier protein. Processes for making particles for delivery of drugs are provided, e.g., in International Patent Publication No. WO2010/036211 and US Patent Publication No. 2009/0196933. International Patent Publication No. WO2005/102507 provides a method for the production of nanoparticles from oil-in-water nanoemulsions prepared by phase inversion techniques. International Patent Publication No. WO2008/032327, herewith incorporated by reference in its entirety as if fully described herein, discloses nanoparticles of a water-insoluble organic compound in the form of redispersible powder or aqueous dispersion, and a process for the production of such nanoparticles from microemulsion. None of these publications teach or suggest nanoparticles comprising compounds having both an NO donor moiety and an ROS degradation catalyst moiety.

Whereas compounds of the general formula I such as compound 1a particularly exemplified herein are soluble in ethyl acetate and DMSO, they are insoluble in non-toxic aqueous liquids suitable for human administration. Accordingly, a method of providing such compounds in a liquid compatable with human use is desirable so as to allow for clinical delivery by an intravenous, subcutaneous, or intratracheal route. Examples 2-4 hereinafter show the preparation of dispersible powders comprising nanoparticles comprising compound 1a. For the preparation of those powders, oil-in-water microemulsions of compound 1a together with one or more surfactants and optionally sucrose were first prepared and then lyophilized. The dispersible powders prepared contained about 18-25% (by weight) of compound 1a and about 60-80% (by weight) of surfactants, and were easily dispersible in water and in isotonic solution of dextrose up to 5% by weight, producing stable translucent suspensions of up to 2 mg of the active agent/ml, which are readily sterile-filtered via a 0.22-μ filter and are well tolerated when injected parenterally into rodents. The majority of the resulting nanoparticles had an average size of 80 nm, as determined by number distribution in light scattering measurements. Such nanoparticles have better dissolution rate and solubility than conventional microparticles, and may thus provide enhanced bioavailability of the active agent.

In a further aspect, the present invention thus provides a water dispersible powder comprising nanoparticles comprising the active agent, i.e., a compound of the general formula I as defined above, or an enantiomer, diastereomer, racemate, or pharmaceutically acceptable salt or solvate thereof.

The term “nanoparticles” or “nanoparticulate” as used herein describes particles having an average diameter of about 1 nm to about 1000 nm. Preferably, the nanoparticles have a diameter of about 350 nm or less, more preferably less than 300 nm, most preferably less than about 250 nm. In particular embodiments, the nanoparticles have a diameter in the range of 10-500 nm, 20-350 nm, 30-300 nm, 40-250 nm or 50-200 nm.

The term “dissolution rate” as used herein describes the relative dissolution rate of a solute in a solvent, more particularly, the relative time required to dissolve specific proportions of a solvent and a solute required to affect dissolution of the solute in the solvent. The solubility of the nanoparticles is defined as the concentration of the active agent in an aqueous solution after filtering the dispersion through a 0.22-μ filter. As described herein, having the active agent in the form of nanoparticles can significantly increase solubility and dissolution rate as compared to the same compound in an unprocessed form, i.e., in a form that has not undergone any particle size reduction or other treatment to increase its solubility or dissolution rate. In certain embodiments, the solubility of the active agent, i.e., the concentration of the active agent in a liquid filtered by a 0.22-μ filter, when formulated as nanoparticles is at least about 5 times, preferably about 10 times, greater than its solubility in an unprocessed form. In other embodiments, the dissolution rate of the active agent when formulated as nanoparticles is at least about 5 times, preferably about 10 times, greater than its the dissolution rate in an unprocessed form.

In certain embodiments, the water dispersible powder of the present invention comprises nanoparticles as defined above, wherein said nanoparticles further comprise at least one surfactant, and optionally a polymer, preferably a non-cross-linked polymer that is acceptable for administration to humans. In particular embodiments, said at least one surfactant is a cationic surfactant, an anionic surfactant, an amphoteric surfactant, a nonionic surfactant, or a polymeric surfactant.

The surfactant comprised within the water dispersible powder of the invention is a surface-active agent, which increases the emulsifying, foaming, dispersing, spreading and wetting properties of the powder, and is further acceptable for administration to humans. Examples of cationic surfactants include, without being limited to, hexyldecyltrimethylammonium bromide, and hexyldecyltrimethylammonium chloride; non-limiting examples of anionic surfactants include sodium dodecyl sulfate, sodium sulfosuccinate, sodium stearate, sodium oleate, ammonium glycyrrhizinate, dipotassium glycyrrhizinate, dicalcium glycyrrhizinate, a cholate, a deoxycholate such as sodium deoxycholate, and mixtures thereof; examples of amphoteric surfactants include, without being limited to, a lecithin such as egg lecithin and soybean lecithin, a synthetic saturated lecithin such as dimyristoyl phosphatidylcholine, dipalmitoyl phosphatidylcholine and distearoyl phosphatidylcholine, a synthetic unsaturated lecithin such as dioleyl phosphatidylcholine and dilinoleyl phosphatidylcholine, a pegylated phospholipids, and mixtures thereof; examples of nonionic surfactants include, without limiting, a polysorbate such as polyethylene sorbitan monooleate, an ethoxylated sorbitan ester, sorbitan ester, polyglycerol ester, sucrose ester, alkyl polyglucoside, polyalkyleneoxide modified heptamethyltrisiloxane, allyloxypolyethylene glycol methylether, saponin, and mixtures thereof; and non-limiting examples of polymeric surfactants include poloxamer, polyvinyl alcohol, gum Arabic, chitosan, and mixtures thereof.

Examples of polymers include, without limiting, polylactic acid, cellulose acetate, methyl cellulose, hydroxylpropyl methyl cellulose, poly(lacticco-glycolic acid), hydroxylpropyl cellulose phthalate, polyvinyl pyrrolidone (PVP), carboxy methyl cellulose, hydroxy ethyl cellulose, polyethylene glycol, polylysine, alginate, and mixtures thereof.

In certain embodiments, the water dispersible powder of the present invention comprises about 10%, 15%, 20%, 25%, 30%, 35%, 40%, or more, by weight of the active agent, about 40%, 50%, 60%, 70% or 80% by weight of said at least one surfactant, and optionally up to 10%, 20%, 30%, 40% or 50% by weight of said polymer.

In certain embodiments such as exemplified herein, the water dispersible powder comprises the active agent as well as (i) polyethylene sorbitan monooleate, soybean lecithin, and sucrose; (ii) sodium deoxycholate, and soybean lecithin; or (iii) ammonium glycyrrhizinate, and soybean lecithin.

In specific embodiments, the active agent comprised within the water dispersible powder of the invention is selected from compounds 1a, 1b, 2a, 2b, 3a, 3b, 4a, 4b, 5a, 5b, 6a, 6b, 7a, 7b, 8a, 8b, 9a, 9b, 10a, 10b, 11a, 11b, 12a, 12b, 13a, 13b, 14a, 14b, 15a, 15b, 16a, 16b, 17a, 17b, 18, 19 or 20, preferably compound 1a, or an enantiomer, diastereomer, racemate, or pharmaceutically acceptable salt or solvate thereof.

The water dispersible powder of the present invention, as well as aqueous dispersions comprising thereof can be prepared according to any suitable procedure known in the art, e.g., as described in WO 2008/032327.

In certain embodiments, the water dispersible powder of the invention is prepared as shown in the Examples herein, i.e., by a process comprising the steps of (i) preparing an oil-in-water microemulsion comprising the active agent, a volatile water-immiscible organic solvent, water, said at least one surfactant, and optionally said polymer; and (ii) removing the volatile water-immiscible organic solvent and the water thus forming the desired dispersible powder. In particular embodiments, the oil-in-water microemulsion is prepared by dissolving the active agent in the volatile water-immiscible organic solvent to form an organic phase, and mixing the organic phase with the water, the surfactant, and optionally the polymer. In other particular embodiments, the volatile water-immiscible organic solvent and the water are removed, either simultaneously or sequentially in any order, by reduced pressure, lyophilization or spray drying.

The term “microemulsion” as used herein refers to both oil-in-water as well as “reverse”, i.e., water-in-oil, microemulsions. An oil-in-water microemulsion is a translucent to transparent dispersion of an organic phase in an aqueous phase, having a droplet diameter size in the nanometer range. Whereas oil-in-water emulsions having droplets of larger diameter can be thermodynamically unstable and/or require high shear forces to induce their formation, the oil-in-water microemulsion referred to herein is thermodynamically stable and is generally spontaneously self-emulsifying upon mixture of appropriate surfactant(s), cosurfactant(s), solvent(s), cosolvent(s), water insoluble material and water. A water-in-oil microemulsion is a translucent to transparent dispersion of an aqueous phase in an organic phase, which is thermodynamically stable.

The oil-in-water microemulsion prepared in step (i) of the aforesaid process is a dispersion or emulsion of droplets of a water-insoluble volatile organic solvent in an aqueous medium, with the droplets having an oily core dissolving the active agent, surrounded by an interfacial film of at least one surfactant. The emulsification process denotes the formation of the droplets dispersed within the aqueous phase. The oil-in-water microemulsion comprises the active agent, a volatile water-immiscible organic solvent, water, at least one surfactant, and optionally a polymer. The microemulsion may further comprise a dispersion aid, i.e., an agent that promotes dispersion of the powder of the invention within an aqueous solution phase, or a co-solvent. Suitable dispersion aids include, e.g., wetting agents, disintegrants, water-soluble polymers, colloidal silica particles, sugars, mannitol, and mixtures thereof.

In order to prepare the oil-in-water microemulsion, an organic phase and an aqueous phase are separately prepared and are then mixed together. To prepare the organic phase, the active agent is dissolved in a volatile water-immiscible organic solvent, optionally in combination with a co-solvent. The aqueous phase is prepared by combination of the aqueous components, usually including the surfactant(s) and water, and optionally in combination with a polymer and/or dispersion aid. Alternatively, the surfactant(s) and optionally the polymer and/or the dispersion aid are mixed in the organic phase. Such dissolution steps can be spontaneous or can be carried out using various mechanical stirring instruments. The temperature and length of time for carrying out the dissolution steps can be adjusted as required to achieve improved results. The respective organic and aqueous phases so obtained are then mixed together to obtain a microemulsion, which is formed spontaneously by simple mechanical means such as vortexing.

The volatile water-immiscible organic solvent used in step (i) of the process defined above is effective for dissolution of the active agent. Furthermore, said organic solvent is volatile at the concentration used, such that it can be removed from the oil-in-water microemulsion in the second step of the process. The volatile water-immiscible organic solvent should be suitable for administration to humans in trace amounts. Non-limiting examples of appropriate volatile water-immiscible organic solvents include n-butyl acetate, sec-butyl acetate, isobutyl acetate, propyl acetate, toluene, xylenes, R(+)-limonene, hexane, pentane, heptane, and mixtures thereof.

Alternatively, dissolution of the active agent can be achieved using the volatile water-immiscible organic solvent in combination with a co-solvent that is either miscible or immiscible with water, and is suitable for administration to humans in trace amounts. Examples of suitable co-solvents include, without limiting, ethanol, 1-propanol, 2-propanol, n-pentanol, n-butanol, ethyl acetate, propylene glycol, glycerol, polyethylene glycol, and mixtures thereof. In certain embodiments, the co-solvent is present in an amount of about 5 to about 30% by weight based on the total weight of the microemulsion.

In still a further aspect, the present invention provides a pharmaceutical composition comprising a water dispersible powder as defined above and a pharmaceutically acceptable carrier or diluent. In certain embodiments, the nanoparticles comprised within this composition are in a particulate form, i.e., discrete, individual, non-aggregated particle entities composed of the active agent, such that the active agent is not enclosed within, incorporated within, embedded within, contained within or associated with any encapsulation form, bead, carrier, matrix or similar delivery agent. In certain embodiments, the composition is formulated as a dispersible powder, a tablet, a capsule, a granule, a bead, an aqueous dispersion, an aerosol, or a suspension. A dispersible powder provides a product having a long shelf life and possessing minimal bulk and weight properties as compared to a liquid form. A dispersible powder can be converted, if desired, to an aqueous dispersion upon contact with an aqueous medium such as water, saline, or other isotonic solution. In particular embodiments, the composition is for intravenous, intramuscular, subcutaneous, inhalation, or intratracheal administration.

Aqueous dispersion compositions can be prepared by a process similar to that defined above for the preparation of the water dispersible powder. According to this process, an oil-in-water microemulsion comprising the active agent, a volatile water-immiscible organic solvent, water, at least one surfactant, and optionally a polymer is first prepared, and the volatile water-immiscible organic solvent is then removed so as to form the desired aqueous dispersion. Alternatively, an oil-in-water microemulsion is prepared by first dissolving the active agent in the volatile water-immiscible organic solvent so as to form an organic phase; and then mixing the organic phase with water, at least one surfactant, and optionally a polymer so as to spontaneously form the oil-in-water microemulsion.

In yet a further aspect, the present invention relates to a method of prevention, treatment or management of PH in an individual in need thereof, comprising administering to said individual a pharmaceutical composition comprising a water dispersible powder as defined above and a pharmaceutically acceptable carrier or diluent.

The invention will now be illustrated by the following non-limiting Examples.

### EXAMPLES

### Materials and Methods

### Experimental Design

Adult male Sprague-Dawley rats (250-350 g) (3 groups; n=5 per group) were treated with a single subcutaneous injection of monocrotaline (MCT; 60 mg/kg), a plant poison that induces a well-characterized experimental model of pulmonary hypertension, or an equivalent volume of saline (2 ml/kg; control). After a period of 38 days in which rats developed severe pulmonary arterial hypertension (PAH), dosing with compound 1a for 10 days was initiated, as follows: group 1 (sham animals) did not receive MCT; group 2 was dosed with vehicle control in drinking water; group 3 was dosed with compound 1a 1.5 mg/kg/day BID (twice a day) via an IP route; and group 4 was dosed with compound 1a 2 mg/kg/day via drinking water. At the conclusion of the 10-day dosing period, rats were anesthetized and instrumented, and resting hemodynamic indices were recorded.

### Blood Pressure Measurements

Rats were anesthetized with intramuscular ketamine (90 mg/kg) and pentobarbital sodium (15 mg/kg). The trachea was exposed and cannulated with a plastic tube that was connected to a Harvard ventilator. The animals were ventilated with room air at a tidal volume of 1.5 ml at a rate of 100 breaths/minute. A polyethylene catheter (PE-50) was inserted into the right carotid artery to measure the mean systemic arterial pressure (SAP). A polyvinyl (PV-1) catheter was inserted through the right jugular vein via the right atrium and ventricle into the pulmonary artery for measurement of the mean pulmonary arterial pressure (MPAP). Haemodynamic variables were measured with a pressure transducer and recorded on MacLab A/D converter (AD Instruments), and stored and displayed on a Macintosh personal computer.

### Optical Microscopy

The lower lobe of the right lung was fixed with formalin solution. After paraffin embedding, 5 mm sections were stained with haematoxylin and eosin and observed in a Dialux 22 Leitz (Wetziar, Germany) microscope. The score of lung fibrosis was assessed on sections stained with Masson Trichrome staining. For morphometric evaluations, all three lobes of right lung were inspected. For each lobe the vessels of medium and small size that demonstrated edema and inflammatory cells were counted. Results are expressed as the percentage of vessels presenting indices of disease relative to the total number of vessels counted in the sections. The percentage of vessels demonstrating thickening of the layer of smooth muscle in the tunica were also expressed as a percentage relative to the total number of vessels counted.

### Example 1

### The Effect of R100 on MCT-Induced Changes in Systemic and Pulmonary Arterial Pressure, and on MCT-Induced Pulmonary Vascular Remodeling

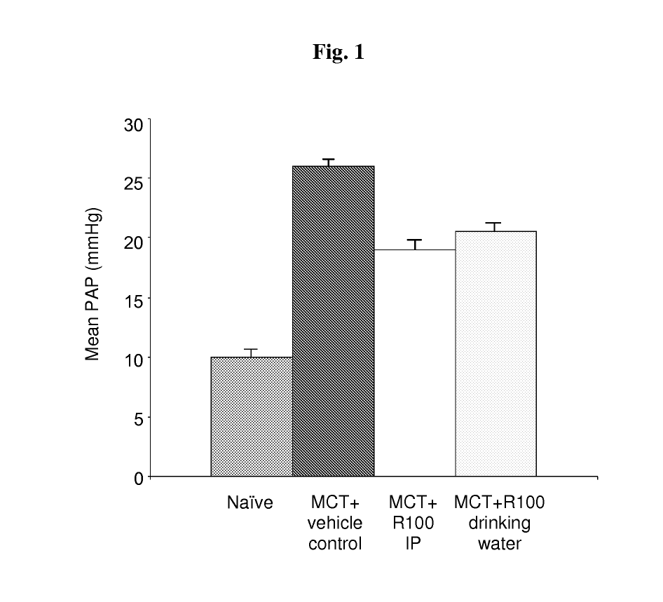

Chronic dosing of compound 1a (for 10 days) was highly effective in reducing the elevation of pulmonary hypertension (PH). As shown in FIG. 1, the mean pulmonary arterial pressure (MPAP) in the rats treated with MCT and vehicle control (group 2) was significantly elevated compared with sham-treated rats (group 1), whereas chronic treatment with compound 1a significantly reduced the elevation of MCT-induced MPAP by about 50% (group 3 and 4).

Compound 1a was well tolerated, as noted by an absence of any effect on body weight or activity level.

TABLE 1
Compound 1a affects MCT-induced histological alterations in the lung

| | | | | Perivascular | Muscolaris |
|---|---|---|---|---|---|
| | Fibrosis | Alveolar | Angioedema | infiltrate | thickening |
| Group¹ | score | damage | score² | score² | score² |
| 1 | 0.00 ± 0.00 | 0.00 ± 0.00 | 0.00 ± 0.00 | 0.00 ± 0.00 | 0.00 ± 0.00 |
| 2 | 3.30 ± 2.87 | 2.80 ± 0.87 | 24.30 ± 4.85 | 40.43 ± 23.48 | 7.61 ± 1.97 |
| 3 | 0.80 ± 0.57 | 0.50 ± 0.35 | 10.00 ± 8.86 | 5.95 ± 5.71 | 0.00 ± 0.00 |
| 4 | 1.00 ± 0.79 | 0.70 ± 0.45 | 7.00 ± 2.78 | 6.06 ± 2.44 | 0.00 ± 0.00 |

¹Group 1 (sham); Group 2 (MCT + vehicle, PO); Group 3 (MCT + compound 1a, 1.5 mg/kg/day BID, IP); Group 4 (MCT + compound 1a, 2 mg/kg/day BID, PO)
²Expressed as the percentage of vessels presenting indices of disease relative to the total number of vessels counted in the sections

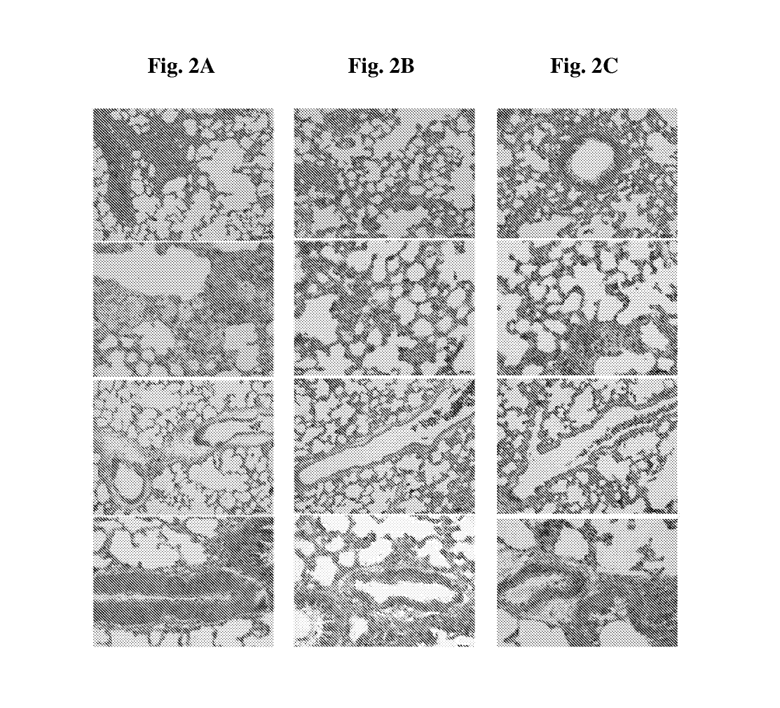

As summarized in Table 1 and shown in FIGS. 2A-2C, no histological alterations were observed in the lung tissues from sham-treated rats. Hematoxylin-eosin staining of lungs of rats treated with MCT (group 2) showed diffuse alveolar damage, interstitial edema with thickened alveolar septae, perivascular edema, and inflammatory cell infiltration. There was no evidence of pulmonary edema, but some areas of the lung developed fibrotic foci accompanied by inflammatory cell infiltration (lymphocytes and granulocytes). The layer of vascular smooth muscle was affected and there were signs of adventitial and perivascular edema. Small pulmonary arteries showed no obvious signs of muscularization in fibrotic areas (FIG. 2A). Chronic treatment with compound 1a (groups 3 and 4) significantly reduced the alveolar damage, the inflammatory cell infiltrate, and the vascular smooth muscle hypertrophy as compared to the vehicle control (group 2). The chronic oral treatment with enteral compound 1a (FIG. 2C) was slightly more protective than with IP treatment (FIG. 2B). The results demonstrate that compound 1a reduces histological injury and diminishes the elevation in MPAP by 50%. This finding is in the setting wherein the onset of compound 1a therapy was delayed for 38 days after the injection of MCT, i.e. therapy was begun at a timepoint of established PH and lung injury. The currently marketed endothelin receptor antagonist, bosentan, has been reported in the literature to have no effect in this same rodent model system when the initiation of therapy was comparably delayed as in this study of compound 1a.

### Example 2

### Preparation of Dispersible Powder Comprising Nanoparticles of Compound 1a

An oil-in-water microemulsion was prepared having the indicated percent weight proportions of the following materials: polyoxyethylene sorbitan monooleate (Tween-80™; a nonionic surfactant; 11.3%), soybean lecithin (a surfactant; 11.3%), n-butyl acetate (12.1%), ethanol (19.3%), sucrose (6.5%), water or phosphate buffer pH=7 (33.0%) and compound 1a (6.5%).

In order to prepare the microemulsion, the required quantity of compound 1a was first dissolved in the mixture of n-butyl acetate and ethanol, and Tween-80 and soybean lecithin were then dispersed in the resulting solution to prepare an organic phase. Next, sucrose was dissolved in either water or phosphate buffer to prepare an aqueous phase, and the aqueous and organic phases were then mixed together and vortexed until a transparent microemulsion was formed.

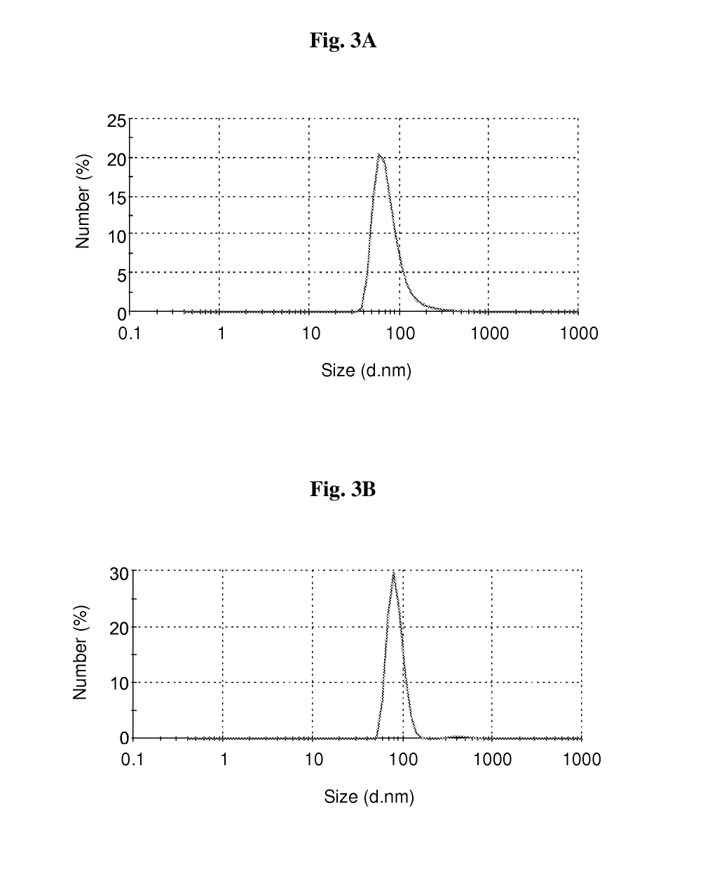

The microemulsion obtained was lyophilized and the resulting dispersible powder contained 18.3% compound 1a by weight, as well as 18.3% sucrose, 31% lecithin and 31% Tween-80. The powder was easily dispersible in water or in isotonic solution of dextrose (5 wt %) up to 5% by weight. The majority of the resulting nanoparticles had an average size of 80 nm, as determined by number distribution in light scattering measurements. FIGS. 3A-3B show graphs demonstrating the particle size distribution of the powder prepared when dispersed in water (3A) and in isotonic dextrose solution (3B).

### Example 3

### Preparation of Dispersible Powder Comprising Nanoparticles of Compound 1a

An oil-in-water microemulsion was prepared having the indicated percent weight proportions of the following materials: sodium deoxycholate (a surfactant; 10%), soybean lecithin (a surfactant; 10%), n-butyl acetate (15%), sec-butyl alcohol (20%), water or phosphate buffer pH=7 (40%) and compound 1a (5%).

In order to prepare the microemulsion, the required quantity of compound 1a was first dissolved in the mixture of n-butyl acetate and sec-butyl alcohol, and sodium deoxycholate and soybean lecithin were then dispersed in the resulting solution to prepare an organic phase. Next, water (or buffer) was added to the organic phase, and the system was then vortexed until a transparent microemulsion was formed.

The microemulsion obtained was lyophilized and the resulting dispersible powder contained 20% compound 1a by weight, as well as 40% lecithin and 40% sodium deoxycholate. The powder was easily dispersible in water or in isotonic solution of dextrose (5 wt %) up to 5% by weight.

### Example 4

### Preparation of Dispersible Powder Comprising Nanoparticles of Compound 1a

An oil-in-water microemulsion was prepared having the indicated percent weight proportions of the following materials: ammonium glycyrrhizinate (a surfactant; 10%), soybean lecithin (a surfactant; 10%), n-butyl acetate (14%), sec-butyl alcohol (10%), water or phosphate buffer pH=7 (50%) and compound 1a (6%).

In order to prepare the microemulsion, the required quantity of compound 1a was first dissolved in the mixture of n-butyl acetate and sec-butyl alcohol, and ammonium glycyrrhizinate and soybean lecithin were then dispersed in the resulting solution to prepare an organic phase. Next, water (or buffer) was added to the organic phase, and the system was then vortexed until a transparent microemulsion was formed.

The microemulsion obtained was spray dried and the resulting dispersible powder contained 23% compound 1a by weight, as well as 38.5% lecithin and 38.5% ammonium glycyrrhizinate. The powder was easily dispersible in water or in isotonic solution of dextrose (5 wt %) up to 5% by weight.

### REFERENCES

Galiè N., Torbicki A., Barst R., Dartevelle P., Haworth S., Higenbottam T., Olschewski H., Peacock A., Pietra G., Rubin L. J., Simonneau G., Priori S. G., Garcia M. A., Blanc J. J., Budaj A., Cowie M., Dean V., Deckers J., Burgos E. F., Lekakis J., Lindahl B., Mazzotta G., McGregor K., Morais J., Oto A., Smiseth O. A., Barbera J. A., Gibbs S., Hoeper M., Humbert M., Naeije R., Pepke-Zaba J., Guidelines on diagnosis and treatment of pulmonary arterial hypertension. The task force on diagnosis and treatment of pulmonary arterial hypertension of the European Society of Cardiology, Eur Heart J., 2004, 25, 2243-2278
McCrory and Lewis, Methodology and grading for pulmonary hypertension evidence review and guideline development, Chest, 2004, 126, 11-13
McGoon M., Gutterman D., Steen V., Barst R., McCrory D. C., Fortin T. A., Loyd J. E., Chest, 2004, 126, 14S-34S

1. A method for prevention, treatment or management of pulmonary hypertension (PH) in an individual in need thereof, comprising administering to said individual a therapeutically effective amount of a compound of the general formula I: 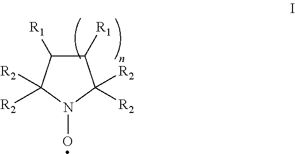
or an enantiomer, diastereomer, racemate, or pharmaceutically acceptable salt or solvate thereof,
wherein
R₁ each independently is selected from the group consisting of H, —OH, —COR₃, —COOR₃, —OCOOR₃, —OCON(R₃)₂, —(C₁-C₁₆)alkylene-COOR₃, —CN, —NO₂, —SH, —SR₃, —(C₁-C₁₆)alkyl, —O—(C₁-C₁₆)alkyl, —N(R₃)₂, —CON(R₃)₂, —SO₂R₃, —S(═O)R₃, and an NO-donor group of the formula —X₁-X₂-X₃, wherein X₁ is absent or selected from the group consisting of —O—, —S— and —NH—; X₂ is absent or is (C₁-C₂₀)alkylene optionally substituted by one or more —ONO₂ groups and optionally further substituted by a moiety of the general formula D: 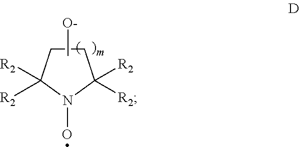
and X₃ is —NO or —ONO₂, provided that at least one R₁ group is an NO-donor group;
R₂ each independently is selected from the group consisting of (C₁-C₁₆)alkyl, (C₂-C₁₆)alkenyl, and (C₂-C₁₆)alkynyl;
R₃ each independently is selected from the group consisting of H, (C₁-C₈)alkyl, (C₃-C₁₀)cycloalkyl, 4-12-membered heterocyclyl, and (C₆-C₁₄)aryl, each of which other than H may optionally be substituted with —OH, —COR₄, —COOR₄, —OCOOR₄, —OCON(R₄)₂, —(C₁-C₈)alkylene-COOR₄, —CN, —NO₂, —SH, —SR₄, —(C₁-C₈)alkyl, —O—(C₁-C₈)alkyl, —N(R₄)₂, —CON(R₄)₂, —SO₂R₄, or —S(═O)R₄;
R₄ each independently is selected from the group consisting of H, (C₁-C₈)alkyl, (C₃-C₁₀)cycloalkyl, 4-12-membered heterocyclyl, and (C₆-C₁₄)aryl; and
n and m each independently is an integer of 1 to 3.

2. The method of claim 1, wherein R₁ each independently is selected from the group consisting of H, —COOR₃, —CON(R₃)₂, and an NO-donor group; and R₃ is H.

3. The method of claim 1, wherein R₂ each independently is (C₁-C₈)alkyl, preferably (C₁-C₄)alkyl, more preferably (C₁-C₂)alkyl, most preferably methyl.

4. The method of claim 3, wherein R₂ are identical.

5. The method of claim 1, wherein in said NO-donor group, X₁ is absent or —O—; X₂ is absent or (C₁-C₂₀)alkylene, preferably (C₁-C₆)alkylene, more preferably (C₁-C₃)alkylene, most preferably methylene; X₃ is —NO or —ONO₂, preferably —ONO₂; and said alkylene is optionally substituted by one or more —ONO₂ groups and optionally further substituted by a moiety of the general formula D.

6. The method of claim 1, wherein (i) n is 1; and one or two of the carbon atoms at positions 3 or 4 of the pyrrolidine ring are linked to an NO-donor group; (ii) n is 2; and one or more of the carbon atoms at positions 3 to 5 of the piperidine ring are linked to an NO-donor group; or (iii) n is 3; and one or more of the carbon atoms at positions 3 to 6 of the azepane ring are linked to an NO-donor group.

7. The method of claim 6, wherein said compound comprises more than one identical or different NO-donor groups.

8. The method of claim 6, wherein each one of said NO-donor groups independently is of the formula —(C₁-C₆)alkylene-ONO₂, preferably —(C₁-C₃)alkylene-ONO₂, more preferably —CH₂—ONO₂, or —O—(C₁-C₆)alkylene-ONO₂, wherein said alkylene is optionally substituted by one or more —ONO₂ groups; or is —ONO₂.

9. The method of claim 8, wherein n is 1; R₂ each is methyl; and
(i) R₁ linked to the carbon atom at position 3 of the pyrrolidine ring is the NO-donor group —CH₂—ONO₂ or —ONO₂; and R₁ linked to the carbon atom at position 4 of the pyrrolidine ring is H (herein identified compounds 1a and 1b, respectively); or
(ii) each one of R₁ linked to the carbon atoms at positions 3 and 4 of the pyrrolidine ring is the NO-donor group —CH₂—ONO₂ or —ONO₂ (herein identified compounds 2a and 2b, respectively).

10. The method of claim 8, wherein n is 2; R₂ each is methyl; and
(iii) R₁ linked to the carbon atom at position 3 of the piperidine ring is the NO-donor group —CH₂—ONO₂ or —ONO₂; and each one of R₁ linked to the carbon atoms at positions 4 and 5 of the piperidine ring is H (herein identified compounds 3a and 3b, respectively);
(iv) R₁ linked to the carbon atom at position 4 of the piperidine ring is the NO-donor group —CH₂—ONO₂ or —ONO₂; and each one of R₁ linked to the carbon atoms at positions 3 and 5 of the piperidine ring is H (herein identified compounds 4a and 4b, respectively);
(v) each one of R₁ linked to the carbon atoms at positions 3 and 4 of the piperidine ring is the NO-donor group —CH₂—ONO₂ or —ONO₂; and R₁ linked to the carbon atom at position 5 of the piperidine ring is H (herein identified compounds 5a and 5b, respectively);
(vi) each one of R₁ linked to the carbon atoms at positions 3 and 5 of the piperidine ring is the NO-donor group —CH₂—ONO₂ or —ONO₂; and R₁ linked to the carbon atom at position 4 of the piperidine ring is H (herein identified compounds 6a and 6b, respectively); or
(vii) each one of R₁ linked to the carbon atoms at positions 3 to 5 of the piperidine ring is the NO-donor group —CH₂—ONO₂ or —ONO₂ (herein identified compounds 7a and 7b, respectively).

11. The method of claim 8, wherein n is 3; R₂ each is methyl; and
(i) R₁ linked to the carbon atom at position 3 of the azepane ring is the NO-donor group —CH₂—ONO₂ or —ONO₂; and each one of R₁ linked to the carbon atoms at positions 4 to 6 of the azepane ring is H (herein identified compounds 8a and 8b, respectively);
(ii) R₁ linked to the carbon atom at position 4 of the azepane ring is the NO-donor group —CH₂—ONO₂ or —ONO₂; and each one of R₁ linked to the carbon atoms at position 3, 5 and 6 of the azepane ring is H (herein identified compounds 9a and 9b, respectively);
(iii) each one of R₁ linked to the carbon atoms at positions 3 and 4 of the azepane ring is the NO-donor group —CH₂—ONO₂ or —ONO₂; and each one of R₁ linked to the carbon atoms at positions 5 and 6 of the azepane ring is H (herein identified compounds 10a and 10b, respectively);
(iv) each one of R₁ linked to the carbon atoms at positions 3 and 5 of the azepane ring is the NO-donor group —CH₂—ONO₂ or —ONO₂; and each one of R₁ linked to the carbon atoms at positions 4 and 6 of the azepane ring is H (herein identified compounds 11a and 11b, respectively);
(v) each one of R₁ linked to the carbon atoms at positions 3 and 6 of the azepane ring is the NO-donor group —CH₂—ONO₂ or —ONO₂; and each one of R₁ linked to the carbon atoms at positions 4 and 5 of the azepane ring is H (herein identified compounds 12a and 12b, respectively);
(vi) each one of R₁ linked to the carbon atoms at positions 3 to 5 of the azepane ring is the NO-donor group —CH₂—ONO₂ or —ONO₂; and R₁ linked to the carbon atom at position 6 of the azepane ring is H (herein identified compounds 13a and 13b, respectively);
(vii) each of R₁ linked to the carbon atoms at positions 3, 4 and 6 of the azepane ring is the NO-donor group —CH₂—ONO₂ or —ONO₂; and R₁ linked to the carbon atom at position 5 of the azepane ring is H (herein identified compounds 14a and 14b, respectively); or
(viii) each of R₁ linked to the carbon atoms at positions 3 to 6 of the azepane ring is the NO-donor group —CH₂—ONO₂ or —ONO₂ (herein identified compounds 15a and 15b, respectively).

12. The method of claim 8, wherein n is 1; R₂ each is methyl; R₁ linked to the carbon atom at position 3 of the pyrrolidine ring is the NO-donor group —CH₂—ONO₂ or —ONO₂; and R₁ linked to the carbon atom at position 4 of the pyrrolidine ring is —CONH₂ (herein identified compounds 16a and 16b, respectively).

13. The method of claim 8, wherein n is 2; R₂ each is methyl; R₁ linked to the carbon atom at position 3 of the piperidine ring is the NO-donor group —CH₂—ONO₂ or —ONO₂; R₁ linked to the carbon atom at position 4 of the piperidine ring is —COOH; and R₁ linked to the carbon atoms at position 5 of the piperidine ring is H (herein identified compounds 17a and 17b, respectively).

14. The method of claim 8, wherein n is 2; R₂ each is methyl; R₁ linked to the carbon atom at position 4 of the piperidine ring is the NO-donor group —O—CH₂—CH(ONO₂)CH₂—ONO₂; and each one of R₁ linked to the carbon atoms at positions 3 and 5 of the piperidine ring is H (herein identified compound 18).

15. The method of claim 6, wherein each one of said NO-donor groups independently is of the formula —O—(C₁-C₆)alkylene-ONO₂, wherein said alkylene is substituted by a moiety of the general formula D and optionally further substituted by one or more —ONO₂ groups.

16. The method of claim 15, wherein n is 2; each one of R₁ linked to the carbon atoms at positions 3 and 5 of the piperidine ring is H; and (i) R₁ linked to the carbon atom at position 4 of the piperidine ring is the NO-donor group —O—CH₂—CH(ONO₂)—CH(ONO₂)—CH₂-D, wherein in the general formula D, m is 2, and the oxygen atom is linked to the carbon atom at position 4 of the piperidine ring; and R₂ each is methyl (herein identified compound 19); or (ii) R₁ linked to the carbon atom at position 4 of the piperidine ring is the NO-donor group —O—CH₂—CH(ONO₂)—CH₂-D, wherein in the general formula D, m is 2, and the oxygen atom is linked to the carbon atom at position 4 of the piperidine ring; and R₂ each is methyl (herein identified compound 20).

17. The method of claim 9, wherein compound 1a, or an enantiomer, diastereomer, racemate, or pharmaceutically acceptable salt or solvate thereof, is administered.

18. The method of claim 1, wherein said PH is selected from the group consisting of pulmonary arterial hypertension (PAH), PH associated with a left heart disease, PH associated with a lung disease and/or hypoxemia, and PH due to a chronic thrombotic and/or embolic disease.

19. The method of claim 18, wherein said PAH is idiopathic PAH; familial PAH; PAH associated with collagen vascular disease; PAH associated with congenital heart disorders; PAH associated with HIV infection; PAH associated with venous or capillary diseases; PAH associated with thyroid disorders, glycogen storage disease, Gaucher's disease, hemoglobinopathies, or myeloproliferative disorders; PAH associated with either smoke inhalation or combined smoke inhalation and burn injury; PAH associated with aspiration; PAH associated with ventilator injury; PAH associated with pneumonia; PAH associated with Adult Respiratory Distress Syndrome; persistent PH of the newborn; neonatal respiratory distress syndrome of prematurity; neonatal meconium aspiration; neonatal diaphragmatic hernia; pulmonary capillary hemangiomatosis; or pulmonary veno-occlusive disease.

20. The method of claim 18, wherein said left heart disease is a left sided atrial or ventricular disease, or a valvular diseases; said lung disease is chronic obstructive pulmonary disease, an interstitial lung disease, sleep-disordered breathing, an alveolar hypoventilation disorder, chronic exposure to high altitude, or a developmental lung abnormality; and said chronic thrombotic and/or embolic disease is thromboembolic obstruction of distal or proximal pulmonary arteries, or a non-thrombotic pulmonary embolism.

21-37. (canceled)

38. The method of claim 1, comprising administering to said individual a water dispersible powder comprising nanoparticles comprising said compound.
